(19) United States
(12) Patent Application Publication
(10) Pub. No.: US 2005/0177117 A1
(43) Pub. Date: Aug. 11, 2005

(54) Needle with lateral aperture

(76) Inventors: Peter John Crocker, East Molesey (GB); Mervyn Aubrey Little, Frimley Green (GB); Kevin Russell, Oxford (GB)

Correspondence Address: Ronald B Sherer;Bartlett & Sherer, 103 South Shaffer Drive, New Freedom, PA, 17349, US

(21) Appl. No.: 10/509,692

(22) PCT Filed: Apr. 16, 2003
(86) PCT No.: PCT/GB03/01652

(30) Foreign Application Priority Data
Apr. 16, 2002 (GB) 0208627.0

(51) Int. Cl. A61M 5/32

(52) U.S. Cl. 604/272; 600/564; 606/167

(57) ABSTRACT

An improved needle for use with injectors such hypodermic needles have an aperture in its side so that, after the needle penetrates the tissue, the injected fluid is injected via the aperture.

The present invention relates to an improved needle design for use with injection devices such as hypodermic needles.

Hypodermic needles are constructed so that they comprise a hollow tube with an orifice at a sharpened end which penetrates the skin of the person, animal or other organism being injected, normally the sharpened end of the needle is at an angle to the axis of the tube. In use the end of the needle penetrates the skin with the sharpened end cutting through the skin and subcutaneous layers. The size of the needle used depends on the characteristics of the fluid to be injected, with larger diameter needles being used for larger volumes, higher viscosities, higher particulate size and concentration, and for fluids requiring either high flow rate or low shear or low pressure drop.

When using a hollow needle cannula for the conveyance of fluid particularly through the skin of a patient, a number of significant factors must be considered in the design of such cannula. For instance, the needle cannula should be sufficiently rigid and stiff so that it can effectively penetrate the skin of the patient without breaking or bending to such a degree so as to occlude the fluid path. In this regard, such needle cannulae are primarily made of metal so as to impart these desired stiffness characteristics. In addition to surface lubricity of the needle and the sharpness of the point, the outside diameter of the needle and its wall thickness play a factor in the penetration of the skin and the discomfiture attendant with such penetration. It has been suggested that reduction in the outside diameter and the wall thickness of the needle will provide greater ease in penetration of the skin of the patient and less pain and trauma. However, there is usually a tradeoff in merely reducing the outside diameter of the needle in order to achieve this desired ease of penetration. This tradeoff generally involves a narrowing or constriction of the inside diameter of the needle along with the reduction of the outside diameter of the needle. As a result, the flow capacity through the needle is impaired, especially if large flow rates or quantities of fluid are to be conveyed through the needle. Furthermore, increasing the inside diameter or reducing the outside diameter by merely reducing the wall thickness of the needle is limited to a fraction of the wall thickness and will compromise the stiffness characteristics of the needle so that there will be a greater tendency to bend or break during its use.

Many of these problems arise because existing needle designs combine two primary functions in the tip i.e. 1) skin penetration and 2) fluid delivery. Combining these two functions has the disadvantage that it forces compromises which stop the two primary functions being independently optimised.

These problems are exacerbated in hypodermic needles when unusually high viscosity or suspensions with high particulate levels or large particle sizes are used, or when unusually large volumes are used. Typical problems if the bore is too small include very slow injection, needle blockage and difficulty in applying sufficient pressure to the syringe plunger for delivery of the required volume of fluid in an acceptable time.

A further problem arises with pain and trauma to the body tissues at the fluid injection site, due to distension caused by the bolus of larger volumes of injected fluid.

One solution to the fluid flow problems is to use a larger bore needle, but when larger bores are used to overcome the restrictions other problems arise due to pain and trauma of the tissue caused by the needle blade. Also, the larger needles are prone to forming a long cylindrical tissue core inside the bore of the needle during the needle entry into the body tissue. Such tissue cores are left at the injection site where they can necrotise and cause post injection pain for some days. Other needle types are known which have an atraumatic tip to reduce tissue cutting and coring, but these require an additional sharp cutting scalpel or hollow introducer or an autoinjector to achieve entry through the skin.

Needles have been disclosed for reducing these problems in which the needles have a dispensing orifice at one end and a first portion distal from the dispensing orifice and a second portion proximal the dispensing orifice with the outside diameter of the second portion being smaller than the outside diameter of the first portion. U.S. Pat. Nos. 3,216,616, 3,540,447, 3,993,079, 4,335,718, 4,781,691, 5,792,099, and 5,951,528 disclose such “stepped” needles. In all these needles the dispensing orifice is at the sharpened end of the needle and is not positioned on or immediately adjacent to the larger bore section. For this reason, if a significantly smaller bore tip is used the flow resistance to newtonian fluids is exponentially increased, and there is significant probability of blockage by particulate fluids.

Another type of needle U.S. Pat. No. 4,710,180 has lateral holes and a blunt tip, but is not stepped and lacks an elongate taper section. This is for lipojection, a surgical procedure requiring an incision to be made in the skin first, after which the needle is inserted through the incision. This has many of the usual disadvantages of larger bore needles and is not well suited for routine injections.

Another type of needle has one or more small lateral holes along the axis of a 22—29 gauge needle for saline based therapies. U.S. Pat. Nos. 4,411,657 and 6,517,521 disclose such needles. The needles have a pointed tip but are not stepped and lack an elongate taper section. These needles would not be suitable for the more viscous and particulate fluids mentioned above.

We have now devised an improved needle structure which reduces the above problems.

According to the invention there is provided a needle for use with means to inject or remove material comprising (i) a tip which tapers to a point, (ii) an elongate section adjacent the tip, said elongate section comprises a ramp and optionally a parallel section, (iii) a barrel section in fluid connection with the means to inject/withdraw, there being an inner bore comprising a fluidic pathway, which inner bore is within the barrel and optionally extends partially or fully down the elongate section and/or to the tip, there being one or more apertures fluidically connecting the said inner bore to the outside of the needle, and at least part of at least one aperture is lateral or on or adjacent to the barrel and/or the elongate.

By lateral it is meant that the aperture connects the bore to the exterior of the needle through the side of the needle i.e. not through the tip.

The tip may optionally be hollow or solid, sharp or blunt and may optionally include an aperture.

The invention seeks to provide an improved type of hypodermic needle by allowing the two primary functions of the needle (i.e. skin penetration and fluid transfer) to be largely independent and hence enable both to be optimised separately without the disadvantages and compromises forced by existing needle designs. The new needle enables surprising improvements in skin penetration and fluid delivery which provide commercial and therapeutic advantages while meeting the requirements of users and the constraints of commercial manufacture.

The needle can be used with various injection devices such as a hypodermic syringe and other injectors such as those described in patent applications PCT/GB99/02680 and PCT/GB00/03061. In the case of hypodermic injectors the fluid container is the barrel of the syringe or cartridge and the fluid is injected by depressing a plunger as in conventional hypodermic syringes.

The examples below mostly refer to fluid/material injection/insertion, but the needle and related apparatus may also be used for fluid/material withdrawal.

For the sake of clarity, when the needle is being used for injection this patent refers to one or more entry-aperture(s) where the fluid enters the barrel from the syringe or other fluid storage/delivery means. The other one or more apertures through which the fluid exits the needle are simply referred to as aperture(s). This operating mode is distinct from fluid withdrawal through the needle into a syringe (e.g. for blood sampling) which is the opposite of injection hence the exit and entry roles are reversed.

The tip, which is the first part to contact the skin when the needle is inserted into the skin can be a cutting blade, pointed or blunt. The tip is preferably designed for easy penetration of skin and/or tissue with minimum pain, damage or trauma.

Preferably the needle tip is formed from a tube or from a solid cylinder of outside diameter 0.2 to 5.0 mm and the total needle length is 2 to 200 mm. The needle tip diameter is preferably 1% to 99% of barrel diameter and/or the ratio of the tube diameter from which the tip is ground or formed to the barrel diameter is between 1:1.01 and 1:100.

The barrel is connected to the tip by the elongate section and in one embodiment the elongate section tapers from the tip outwardly to the barrel. In another embodiment the elongate section tapers outwardly from the tip for a first section and then has the same diameter as the barrel. Preferably there is an aperture on the tapered section of the elongate section which may extend into the barrel.

Preferably the ratio of the fluidic capacity of the barrel to the fluidic capacity of the tube size equivalent to the tip is between 1.05:1 to 1000:1 and more frequently is 1.05:1 to 200:1.

The flow resistance from the tip end of the barrel to the aperture exit is below 50% and preferably below 10% of the resistance along the barrel.

The flow path from the end of the ramp to the aperture is below 5 mm and preferably below 3 mm.

Preferably the tip and or the rest of the needle are designed to minimise the friction and/or the force required and/or the pain of insertion into and or withdrawal from skin and tissue. This includes the use of one or more lubricants which bond or stick to the needle sufficiently to retain sufficient lubricating effect throughout the penetration, fluid delivery and withdrawal of the needle. Such lubricants are commercially available for example silicone fluid supplied by Dow Corning Corp. or hydrophilic lubricants for example hydrogels.

In one embodiment the tip is preferably angled to enable a tiny cut to be made with a significant part of the extension of the hole formed by needle being by stretch. Preferably 1% to 99% of the maximum aperture formed in skin in use is by stretch which may be wholly or partly reversible when the needle is withdrawn, and correspondingly 99% to 1% is by cutting). The tip can be hollow or solid, and can have a wall thickness equal or different to the barrel and/or elongate section.

In one embodiment the tip and/or other sections have one or more concave areas in between higher ridges, splines or rails running at least partly in parallel with the needle axis to minimise the surface contact area and/or pressure and/or penetration resistance and/or friction between needle and tissue. At least some of the ridges, splines or rails are preferably smooth, non-cutting and generally prepared and radiused to promote low friction gliding through skin and tissue.

Preferably at least part of the elongate section has one or more gradients where the effective outer dimension increases gradually in a linear or curved manner and/or in one or more steps each step comprising one or more gradients from the tip until it joins the barrel section. The overall angle of step or gradient and the angle and/or radius of the leading and trailing edges of step is preferably chosen to ease entry with minimum force and/or pain/trauma of the skin/tissue being penetrated and the depth requirements of the injection. As the percentage of stretch which enlarges the skin hole beyond the cut entry hole is increased it is found that decreasing the gradient angle is useful in achieving low resistance to needle entry. To maintain a short length and a low entry force it is useful to start with a larger included angle of gradient at the elongate section and decrease the angle towards the barrel section.

For example, the barrel may be 0.8 mm diameter tube. The elongate may comprise a small tube 0.4 mm outside diameter and the tip at the end of the elongate may be 0.4 mm wide with a lancet bevel. The ramp joining the small elongate parallel section/tube to the larger barrel, the ramp average may be 7° included angle. The side aperture may be formed with a radius of 10 mm and may be located adjacent to the ramp and barrel.

In a round needle if a tube is taken and then some of the external material ground away to make a slot/groove or a bevel at the tip—the outside dimension which displaces the skin/tissue is reduced and this is what is meant by “effective outside dimension”, as distinct from the perimeter which could include the outer circumference and the adjoining inner circumference of the bore. The “effective outside dimension” covers both the outside diameter and/or the outer circumference e.g. which can be measured with a piece of fine thread e.g. of wire.

If the elongate section has an inner fluid carrying bore connecting fluidically to an aperture at tip, the outside shape can be of o, c, u, x, shape etc.

All or part of the barrel section can be inside the connection to the fluid container and made such that the majority of the greater than average bore of the needle does not penetrate the skin, but conducts the fluid to the point where the bore does penetrate the skin.

The elongate section can be may be hollow or solid and can have a wall thickness equal or different to the barrel.

The elongate section adjacent to the tip maintains or increases the size of a path in tissue formed initially by the tip, which can ease and direct the flow of fluid at least partly along or around the outside of the needle. Preferably the shape of the section is designed to reduce the frictional force of sliding against the adjacent tissue and is preferably is designed to reduce the fluidic friction of the flow going past the section in use.

When fluid pressure is applied, the elongate section forms all or part of a channel which nucleates or eases and/or directs the passage of at least part of the fluid flow with a smaller pressure drop than if the fluid were constrained inside the bore of the tube in that same location.

The barrel section preferably has a larger effective dimension than the elongate section which enables less internal friction and blockage and hence greater fluidic capacity or fluid carrying ability. In use the barrel is connected directly or indirectly (for example via a luer hub or by flexible tube) to a fluid holding and/or dispensing/withdrawing section, for example a syringe or fluid bag. In use the fluid passes from a fluid holding and or dispensing section to the barrel section and the barrel forms all of or part of the fluidic connection between both one or more apertures and the liquid holding and or dispensing section.

The outer shape of the barrel preferably is designed to seal effectively to the skin and/or tissue when inserted therein and may be smooth and substantially round in cross-section to enable such easy sealing.

The barrel may join to and extend inside a luer lock or fluid holding part such as a syringe, and the part which extends inside may have an effective bore greater than the bore which enters the skin/tissue. The part of the barrel which remains outside the skin in use would normally have a fully enclosed bore.

Preferably the barrel has an outside diameter of 0.2 to 25.0 mm. and the bore and/or outside diameter of the barrel and elongate section and/or tip varies e.g. the bore and/or outside diameter changes at the connection between the barrel and the fluid holding or dispensing part.

The one or more apertures provide a fluidic pathway so that fluid moves from the inner bore to the outside of the needle when a pressure differential occurs. Such aperture(s) may be in one or more locations including the tip, the elongate section, the barrel, or at the junction of two sections.

In one embodiment the one or more apertures may extend in length more than 3 diameters of the adjacent tube and/or have an aspect ratio of more than 5:1 length to depth.

In one embodiment preferably the included angle of the one or more tapers and/or apertures is less than 12° and the aperture(s) are positioned and angled to induce fluid to flow along and adjacent to the elongate section and are positioned and angled and radiused to reduce catching particles in the fluid and to reduce pain and catching, cutting or coring of skin or tissue in use.

The superior fluid carrying ability of the needle can be achieved by the close proximity of the aperture to the barrel, but the aperture can be positioned independently of the tip, i.e. the tip can be directly adjacent or contiguous with the aperture or any distance from it. Preferably at least part of at least one aperture is spaced away from the tip and/or on a section of larger outer dimension than the tip.

Preferably the overall dimension of the aperture is larger than the effective outside dimension of the tip by a factor of 10% or more.

The one or more aperture(s) can be wholly or partly on the barrel which is larger than the elongate section.

The one or more aperture(s) can be longitudinal or radial, round or elongate or a slot or a groove, and can penetrate through one wall or through both walls of the tube.

When the needle is to be used for particulate fluids, in order to prevent blockage of needle, it is important that the particulates are not forced into a rigidly confined and reduced bore or aperture dimension. This is very likely to be a problem with existing tapered or stepped needles. The new needle overcomes this problem because the aperture allows the particles to escape when/before they are forced into the ramp at the end of the barrel. This aspect applies throughout the needle including the part of the barrel where the fluid first meets the constriction at the opening of the barrel (i.e. entry-aperture) which will preferably be an entry-aperture of smaller inner dimension than the bore of the barrel, i.e. the end is not square and the included angle is below 90°, in the range 3°-60°, e.g. 45°. The cutaway part may extend across all or part of the bore.

Preferably this entry-aperture is greater than 0.5 mm across and the entire entry-aperture stands above the needle anchorage in the base of the assembly e.g. hub or syringe and may be of inverted ftunnel shape whereby in use the first bore encountered by the fluid is smaller than the bore of the barrel. The aperture may be parallel or angled to the axis of the needle.

Preferably the aspect ratio of the outer tube diameter to slot length of the entry aperture is greater thanor in range 1:1.1 to 1:50.

The risk of blockage of the needle by the particulates can thus be reduced or avoided as the barrel projects into the injector or syringe so that it is proud above the base of the hub, or syringe or injector. This has the effect that, if any particulates fall to the bottom of the hub/syringe, they do not block the barrel of the needle and in use particulates which have clumped to a size that would block the barrel cannot block or enter the barrel but divert away from the entrance to the barrel. Preferably in use the needle barrel projects a distance of more than 1 mm, more preferably more than 3 mm from its anchorage at the bottom of the hub or syringe or injector barrel and distance from the needle barrel to the adjacent sidewalls of the syringe barrel is more than 0.1 mm. The part of the needle barrel projecting into the syringe or injector can have a slot formed in its side which is all or part of the fluidic connection between the opening of the barrel into the syringe of injector.

Preferably at least one aperture is spaced apart from the tip and is located wholly or substantially on the barrel or where the there is a step or gradient change in the outside diameter e.g. it is located on the gradient where the elongate section tapers out to join the larger barrel or it is located on the elongate section and optionally can or cannot not adjoin the tip.

The distance from the tip to at least one aperture is not fixed but is likely to be in the range 0.1 mm to 100 mm depending on the depth of injection required.

In use, as the end of the needle penetrates the skin and tissue, the skin forms a temporary substantially fluid tight seal over the aperture so the fluid passing down the barrel is constrained to pass down the barrel. Under pressure from the injector the fluid then forms a bolus around the aperture and enters the tissue as in conventional needles. In practice this means that part of the fluidic conduit is defined by the tissue itself so that the effective bore of the conduit is wider/larger than the bore entering through the skin.

The aperture may be any shape and is preferably in the form of a groove or slot in the needle and preferably encompasses up to 1 to 80% of the circumference of the bore of the conduit. In use, as the upper layer of skin is strong and elastic and seals tightly to the smooth upper barrel, the aperture is designed to be below the skin surface and the skin forms a seal above the groove, or slot and the groove or slot forms an opening beneath the skin which minimises leakage of fluid being injected during injection and on withdrawal of needle.

The aperture may be parallel or perpendicular or angled to the axis of the needle.

Preferably the ratio of slot length to elongate section length is greater than or in range 1:1.5 to 1:10.

In another embodiment of the invention the aperture is deformable i.e. under pressure from within the bore the aperture opens up so that it gets wider in use.

The end of the elongate nearest the tip can be closed or it can be open, in the latter case, when the aperture is a groove or slot the groove or slot can go to the tip of the needle or it can be distant from the tip of the needle.

A completely or partly solid needle can be used in which a groove is formed in the side of the solid part of the needle, down which groove the fluid to be injected can flow. In this embodiment the groove can terminate a distance from the tip end of the needle which penetrates the skin so that the tip of the needle can form a skin penetration section or the groove can reach to the end of the needle.

Alternatively the needle can comprise a hollow needle with part of the needle axially removed e.g. by cutting or grinding.

The groove can be formed in a solid or hollow needle by forming or deforming the tube e.g. into a “u”, “c”, “v”, “y” or “x” shape or by grinding away a section of the needle.

In use the tip enters the skin first and the elongate section follows into the skin and displaces tissue along its length and forms a guide path for the fluid. The tip enters the tissue and the fluid is injected and enters the tissue along the length of the aperture.

The elongate section in use, can be hollow or solid, and facilitates and guides nucleation of fluidic path at least partially in a forward direction towards the tip which is followed and then expanded when the fluid is applied from the aperture under pressure. The cross sectional outer shape of the elongate section is designed to minimise trauma and can also facilitate the fluid pathway. It can have 0, 1, 2, 3 or 4 axes of symmetry to form corresponding displacement of tissue along its length e.g. the cross section can be an ellipse, rectangle, star, diamond etc. The elongate section can be straight or tapered, it may also form all or part of the gradient or gradients which blends into the barrel and the tip. The elongate length can be chosen to suit the depth of injection required. For example it can be 0.1-3 mm long for shallow or subcutaneous injections, or longer for deeper injections. It can be a single piece or 2 pieces joined, can be swaged or drawn, can be solid or hollow. This does not have a great effect on the fluid flow resistance because the majority of flow is from the barrel to the aperture which is not at the tip.

The needle structure of the present invention can be used with a balloon catheter which comprises a deformable or elastic sheath which has an aperture or apertures in it and which fits around or encases the needle. The balloon catheter can be used with a hollow needle so that the aperture or apertures connect with the conduit or it can be used with a hollow grooved needle so that the aperture or apertures connect with the grooved path.

It is a feature of the invention that it can enable a needle to be used with a shorter confined bore, i.e. the part of the needle which is in the form of a complete tube is shorter. It also allows use of a larger bore for a proportionally greater part of the needles entire length. As much of the pressure drop is the friction of the fluid in the confined bore of the needle, the shorter the confined bore and the greater the proportional length of the larger bore, the less pressure drop, so faster injections and less pressure required. For drugs containing particulates, the needle is also likely to block, so a shorter bore means less pressure drop and less risk of blockage. Once the needle of the invention is through the skin surface, there is a liquid-tight seal where the needle is in the tight upper layers of the skin. Because the tissue below the skin surface is much more pliable and it easily deforms under even small fluid pressure, if at least part the needle below skin is only a “partial bore” e.g. semi-bore or other partial smaller shapes, then it is much easier for the fluid to get along the track defined by the needle, and the fluid contacts more strata/channels/fissures in the tissue so it is easier for the fluid to disperse into the greater number/area of strata.

Because the fluid is spread over more tissue strata/area, it is likely there is less local stretch hence less pain/trauma even for the faster injection rates which the new needle creates and, because there is easier dispersion of fluid and lower back-pressure of fluid in the tissue, there is less chance of blistering and/or back-leakage of fluid out of the hole in the skin.

Overall the needle design of the present invention enables needles with a smaller tip to achieve the same fluidic or particulate carrying capacity as larger conventional needles at lower pressure, with less risk of blockage and with less pain/trauma/leakage than a conventional hypodermic or autoinjector.

The smaller needle can be a shorter needle and/or have a smaller cross-section or can be a needle tapering along its length either gradually, or in “steps” of smaller diameter.

For many uses the needle has a length of 2 to 200 mm.

In use there is preferably a minimum of cutting and displacement by the needle itself in relation to the fluidic capacity and there is a minimum total dimension (length and or area) of sharp cutting edges and/or points, and minimum level of sharpness in relation to the size and fluidic capacity.

In use the small hole formed in skin and tissue by the tip is smoothly extended along the tip and elongate section with minimum of sharp edges or sharp changes in direction to catch or snag tissue.

The needle can comprise at least one separate component made of one or more different materials and which the separate parts are joined together.

The needle can be made by process steps comprising forming a step in the diameter of a needle tube, closing the tip of needle, forming a primary angle onto tip, processing by mechanical or chemical means to increase the radii of the edges of the tip and all apertures, forming step to make final tip dimensions. Preferably the process includes the steps of providing a surface to reduce friction and/or retain lubricant at the surface. It can include increasing the edge radii of all or part of the tip.

The needle can be used with any injection device such as a syringe, pen, autoinjector, syringe driver, tissue/fluid extraction device and the length of the needle is chosen for the application of the needle. Examples of use are to add or remove substance including intradermal, sub-cutaneous, intra-muscular, intra-venous, into bone, into joint, into eye, into any organ, and for keyhole surgery. The needle can also be used to add or remove substance for medicinal or diagnostic or other purposes for human or animal or other applications.

Typical application examples which would benefit from such a needle include volumes above 0.1 ml and especially above 1 ml in a single subcutaneous or intramuscular injection; volumes of up to 1000's of mls for IV or peritoneal exchanges; viscosities above 10 cps to 100,000 cps; particulates larger than 2 μm to 2 mm diameter and concentrations from 1% up to 50%; solid implants of drugs or biomedical devices or sensors; catheterisation; injection or removal of cells especially for shear sensitive cells e.g. cellular therapy, blood removal, transfusion or dialysis. Possible examples of drugs or treatments available or in development include injections requiring needles typically larger than 30G to 12G size; hormone treatments such as human growth hormone; cancer treatments such as prostate cancer; inflammatory diseases such as arthritis; macromolecules including proteins and antibodies; cosmetic treatments such as collagen, liposuction/lipojection; diagnostic sampling procedures e.g. amniocentesis, biopsy; obstetric IVF procedures; injections requiring especially low trauma such as regional anaesthesia or epidural.

When used with an autoinjector, there is preferably a guide which fits to the autoinjector and which fits round the needle and which directly or indirectly helps the needle run straight along its axis with minimum wobble by restricting the axial or lateral movement of needle and/or liquid holding elements attached to needle. The guide can optionally fit around the needle directly, or it can also guide the needle indirectly by guiding the item to which the needle is fixed, e.g. the drug holding syringe or cartridge. This enables easier needle entry and reduces the pain, trauma and bleeding, which is often problem with autoinjectors especially with large needles.

Preferably the guide restricts needle transaxial or lateral movement in use to below 1-10° e.g. 0.01-1° and 1-3 mm e.g. 0.1-1 mm. The guide is preferably of low friction material and may be a simple hollow cylinder or have one or more sprung elements to maintain close sliding tolerance while allowing for variations in manufactured size with minimum frictions. The guide has minimum contact with moving needle-holding element to reduce friction.

The needle of the present invention enables a lower pressure/time profile compared to standard hypodermic needles and enables higher viscosity, higher volume, greater level and/or size of particulates.

The needle of the invention can provide easy entry into skin and or tissue with low entry force, low trauma and low leakage i.e. it can combine minimal cut/tear with gentle stretch to increase path in skin/tissue). It has high fluidic capacity relative to tip size (i.e. can carry high viscosity/volume/particulates/flow rate with minimum pressure drop or blockage; the fluidic capacity is enhanced by reducing the friction and/or blockage at some or all parts of the needle's fluidic path (entrance aperture, bore and exit aperture). The key determinants are diameter and length of bore, as the pressure drop is proportional length/(diameter⁴), where d is the diameter of the bore.

The surface friction between the fluid and the inside of the bore becomes more important for smaller bores, and is reduced by smooth surfaces and low friction treatments and lubricants and smooth transitions without rough edges. The slot itself has little effect on pressure drop unless it is extremely narrow relative to the bore; however, a slot which is small or has rough or sharp edges is much more likely to be blocked by particulates). The needle also is easy to manufacture at low cost.

The needle including one or more of hub, barrel, elongate, tip and point may comprise one or more components joined, and may be made from one or more materials.

The invention is illustrated in the accompanying drawings in which:—

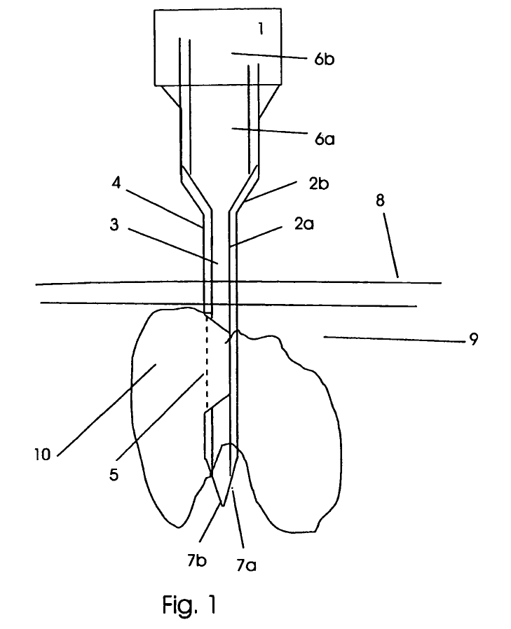

FIG. 1 shows an embodiment of the invention

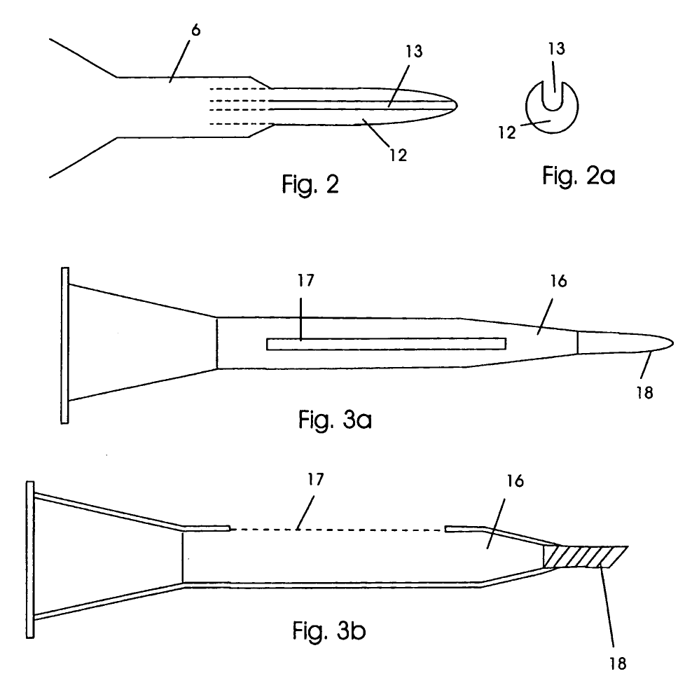

FIGS. 2, 3, 4, 5 and 6 show different embodiments of the invention

FIG. 7 shows cross sectional profiles of grooves which can be used with the needle of FIG. 2

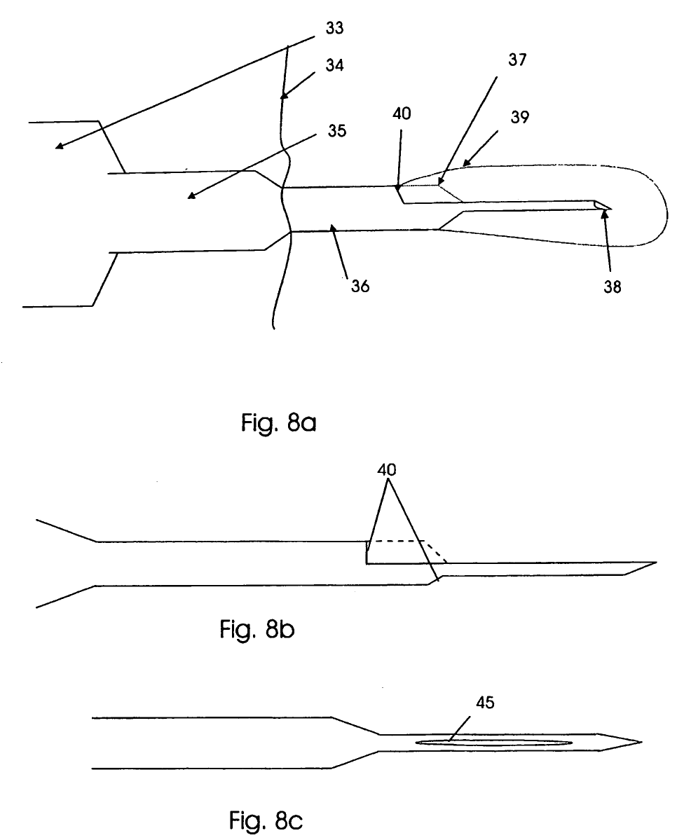

FIG. 8 shows an embodiment of the invention using a stepped needle

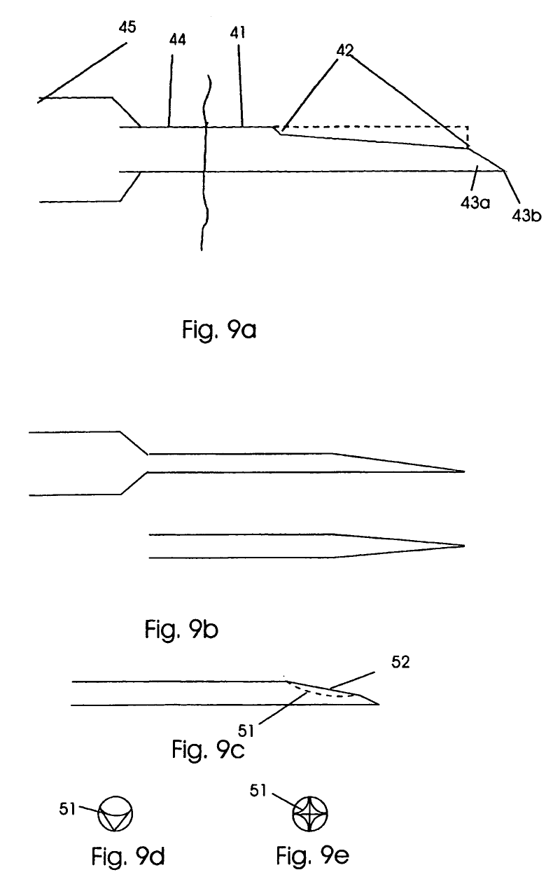

FIG. 9 show other embodiments and

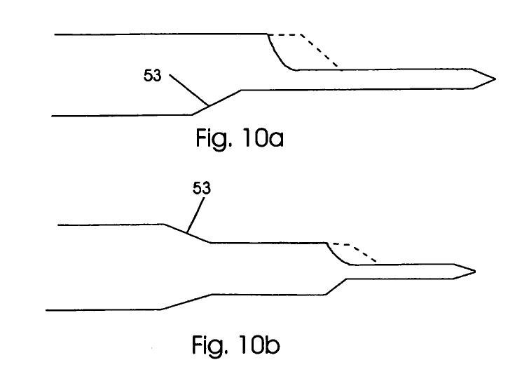

FIG. 10 shows a needle with the gradient away from the heel of the aperture and

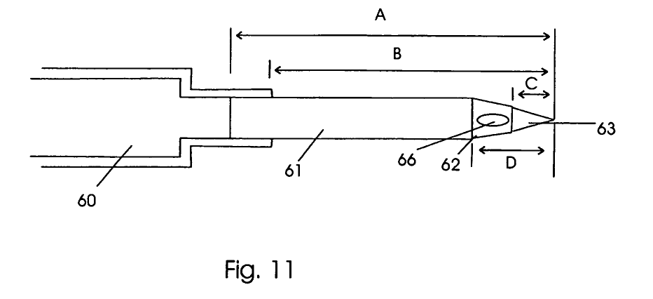

FIG. 11 shows an embodiment of the invention

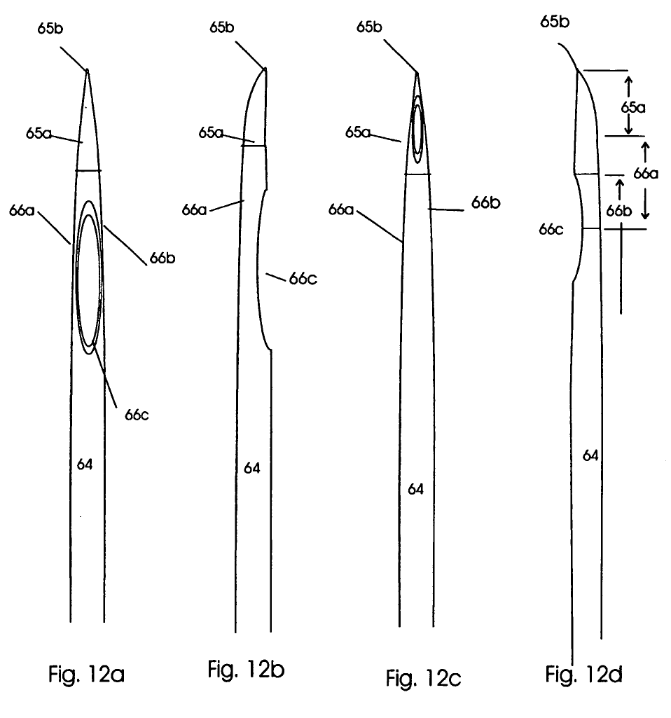

FIG. 12 shows plan and side views of the elongate section and tip of a needle which is another embodiment of the invention and

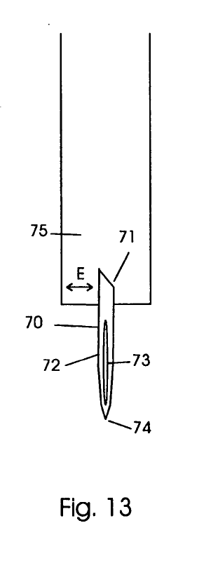

FIG. 13 shows a side view of a modification of the attachment of the needle to a syringe

Referring to FIG. 1 a needle has a tip (7a) with a pointed end (7b), a hub (1) connected to a fluid reservoir and dispenser such as a hypodermic syringe, barrel section (6a) fluidically joined by entry-aperture (6b) connected to the hub and syringe and an elongate section (2a) which includes a ramp (2b) which has an inner bore (3) which is a conduit and which has an outer surface (4). There is a lateral aperture (5) which connects outer surface (4) to the inner bore (3).

In use the needle penetrates the skin (8) which seals on the barrel above the aperture as it passes through it. When the needle has penetrated into the tissue (9) the fluid is injected down inner bore (3) and is injected into tissue (9) through aperture (5) where it forms bolus (10).

As can be seen the aperture and spread of the bolus is wider than would be formed by a conventional needle of the same diameter delivering fluid from the aperture at the tip.

Referring to FIG. 2 this shows a barrel (6) with a bore which enters the skin and an elongate part (12) which is solid or hollow along its length and which has a an aperture in the form of a groove or channel (13) extending from the barrel to the tip end formed in it so that the cross section view of the needle is shown in FIG. 2a In use the needle penetrates the skin and tissue so the barrel end/aperture and groove are below the surface and the fluid is then injected down the groove (13) into the tissue.

FIG. 7 shows various shapes of grooves.

Referring to FIGS. 3a and 3b, these show a plan view and side view of an embodiment in which a needle (16), which can be made of metal or a suitable plastics material, has a slot (17) formed in it, (the slot (17) may extend further than shown). The needle has a tip (18) which can be metal or made of a suitable plastics material and can be blunt or sharp. In use the needle tip (18) penetrates the skin as described above and liquid to be injected passes under pressure down needle (16) and out through slot (17). The slot (17) can be made deformable so that it expands under pressure.

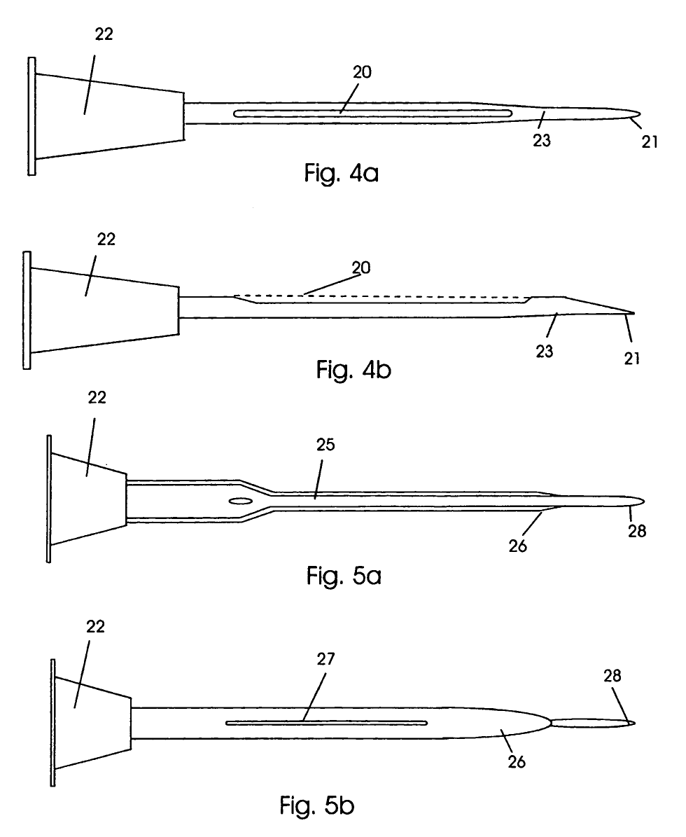

Referring to FIGS. 4a and 4b these show a plan view and a side view of a further embodiment of the invention and in which there is a long slot or opening (20) in needle (23). The needle can be mounted through mounting (22) into a luer or other connector or can be permanently attached to a syringe.

Referring to FIGS. 5a and 5b these illustrate an embodiment being used with a balloon catheter which comprises a deformable or elastic sheath which has an aperture (27) in it which sheath fits around or encases the needle (25) there is a solid or hollow elongate section (28) extending to the tip. In use the tip and all or part of the elongate section enters the skin and tissue and facilitates and guides a path for the needle encased by the balloon catheter to follow. The embodiment of FIG. 5a can also have an even larger bore in the hub.

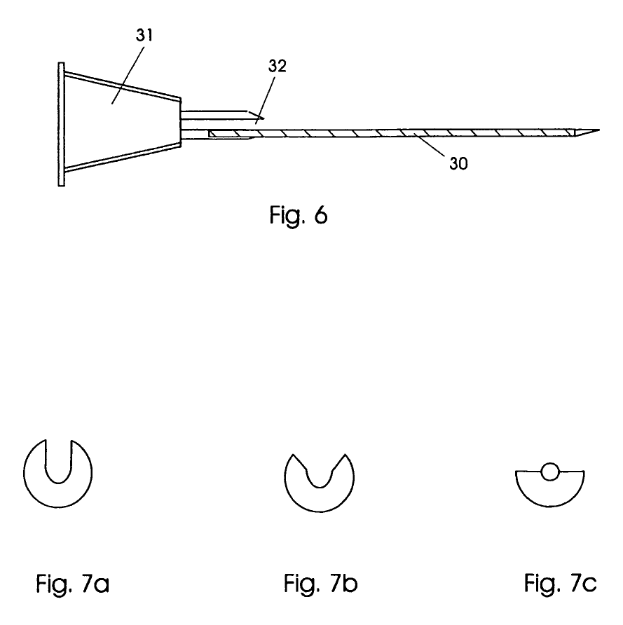

Referring to FIG. 6 there is an off centre solid needle (30) mounted in a needle mounting (31) into a luer or other connector or it can be permanently attached to a syringe. There is a fluid opening (32) which connects to syringe. In use the needle (30) penetrates the skin and tissue and forms a path through the tissue, when fluid is supplied under pressure to opening (32), which is connected to the fluid chamber of a syringe, the path formed by needle (30) facilitates and guides nucleation of fluidic path which is followed and then expanded by the fluid.

Referring to FIG. 8a the syringe body is shown at (33) and the skin is (34). The barrel section has first large bore section (35) and, optionally it can have several reducing sections or a long taper and a second smaller bore section (36). The aperture is formed by cutting away portion (37) of section (36) on or adjacent to the taper or step (40). There is a hollow or solid penetration end (38) which can include an aperture at the tip. In use the end (38) penetrates the skin (34) until the aperture (37) is below the surface and the fluid is then injected and the fluid forms the elongated bolus shown at (39).

Referring to FIG. 8b, this shows a single step or taper from a large bore barrel to the elongate section.

Referring to FIG. 8c, this shows another embodiment in which at least one aperture is in a lateral position entirely on the parallel part of the elongate section away from the tip.

Referring to FIG. 9a there is a syringe (45) connected to barrel section (44), elongate section (41) and tip (43a) with a pointed end (43b). There is a portion of the elongate section (42) cut away to form a slot shaped aperture so the elongate section is tapered, this aperture is adjacent to the barrel and extends to the tip. There is a bore down the inside of the barrel section and elongate section which connects the syringe to the aperture (42).

FIG. 9b shows a side view and a top view of the needle with a tip width approximately 50% Of the barrel and/or elongate outer dimension. The elongate section is a long taper which continues into the tip and end point.

Referring to FIG. 9c this embodiment shows a concave curvature section (51) on the face of tip and/or on other parts of needle and/or ridges, rails or splines the depth of concave is 1-80% of adjacent needle outer diameter; there are rails (52) to the bevel point. FIGS. 9d and 9e show two bevel points and four bevel points respectively when viewed from the tip with concave sections (51).

In use the needle penetrates the skin to location (41) and the syringe operated to inject fluid. As in the other embodiments the skin seals the aperture as it passes through it. When the needle has penetrated into the tissue the fluid is injected down inner bore and is injected into tissue through aperture (42).

Referring to FIG. 10 this shows a needle in which at least part of the gradient (53) is positioned away from the heel of the aperture at the syringe end of the aperture, i.e. opposite end of aperture from the tip.

Referring to FIG. 11 a typical 1-5 ml glass syringe (60) has the barrel (61) of a needle inserted in it. The needle has an elongate section (62) and a tip (63). There is a lateral aperture (66) formed in the elongate section (62) which includes a ramp but has little or no parallel tube section. This helps minimise the length and the resistance to flow and is particularly useful for shallow depth injections below about 5 mm deep e.g. subcutaneous. The total length of the needle is A, length which protrudes from the syringe is B, the length of the tip and elongate section is C and the length of the tip is D.

Referring to FIG. 12a 12b 12c and 12d which show four different views of the needle, FIGS. 12a and 12c show plan views of the needle and FIGS. 12b and 12d show side views, the barrel section (64) adjoins the elongate section (66a) with a ramp (66b) and an aperture (66c) formed in them and has a tip (65a) with a point (65b). The needle ramp, elongate and tip are considerably smaller than the barrel without compromising the fluid flow which gives great benefits to the visual appeal, pain, trauma and usability of the needle.

Referring to FIG. 13 a syringe has a body (75) with a needle (70) connected to the base of the syringe so that a section (71) of the barrel of the needle ends in a non-square entry-aperture e.g. 45° of smaller inner dimension than the bore of the barrel and smoothly radiused which protrudes into the body of the syringe. The needle has an elongate section (72), aperture (73) and a tip (74). There is a space E between the side of the syringe and the needle. In use, when a liquid containing particulates is to be injected any large particles or particles clumping together to be too big to enter the needle are not trapped at the entry-aperture but fall or are diverted into the space E and do not block the needle.

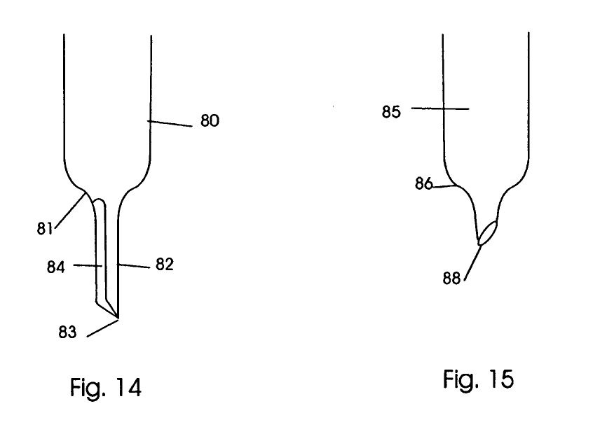

Referring to FIG. 14 the needle has a barrel (80) adjoining an elongate (82) which includes a ramp (81) and aperture (84) leading to the tip (83). At least part of the aperture is lateral, and/or away from the tip, and/or adjacent to the ramp. The ramp (81) can be more than one angle/radius e.g. it can be less steeply angles/radiused towards the barrel and more steeply angled/radiused towards the end with a smaller diameter.

Referring to FIG. 15 the needle has a barrel (85) adjoining an elongate section which includes a ramp (86) and a parallel or tapered tube section (87) leading to a tip (88). The part of the aperture closest to the tip can be spaced from the tip by more than about 0.5 mm. The section (87) is short e.g. below 5 mm long and in use the pressure drop through this section is below 50% of the pressure drop along the barrel.

The use of a needle of the invention is described in the examples.

### EXAMPLE 1

A needle as shown in FIG. 11 of the accompanying drawings with A 24 mm, B 18 mm, C 3 mm and D 5.9 mm made of stainless steel was made and its performance compared with control needles comprising standard Beckton Dickinson Microlance (Registered Trade Mark) needles.

The taper needle device with a 27 gauge tip and a barrel with a 21 gauge outer diameter were selected as the preferred design for in depth testing. This needle is called the 27G/21 needle). The needle was made by taking taper blanks produced using hard tooling. Production scale machines were used to generate the blanks, grinding the tips and forming the apertures. This process was capable of being scaled to produce millions of units. 27G/21 needles produced by these processes were used in the Examples. This showed that the tip entry characteristics, lubrication and fluid flow capacity measured above could be reproducibly produced on commercial scale machinery.

The force required for the 27G/21 needle to penetrate into Melab 0.4 mm thick Polyurethane film was assessed and compared with that of standard 23, 26 and 27G needles.

Test Parameters

Needle velocity 6 mm/s (14.17 inches/minute)
Needles lubricated with a 12500 cps silicone from Dow Corning or similar.
23G, 26G, 27G standard needles such as Beckton Dickinson
Force measured on Mecmesin PF1 200N force indicator
Ambient temperature, 18° C. at 58% RH.
   Film Penetration Force Test—Protocol

The film, not under tension, was clamped flat between two flat plates, each with a 6.75 mm diameter hole (approximately 0.25 inch diameter hole). The needle was held in a small collet attached to Mecmesin force indicator and aligned towards the film at the centre of the hole in the support plates.

To measure penetration force, the force indicator and needle were driven forwards along a linear slide by linear actuator in a direction normal to the flat surface of the test film. When penetration was complete, the linear actuator was reversed and the withdrawal force measured. The maximum and minimum forces were recorded electronically and shown in table 1.

TABLE 1

| | Maximum Tip Force* | |
|---|---|---|
| | (grammes) ± | Maximum Ramp Force^{@} |
| Needle | standard deviation | (grammes) (DepotOne) |
| Taper 27G/21 Needle | 67 ± 5.0 grammes | 72.1 ± 3.5 grammes |
| Standard 27 gauge | 85 ± 8.0 grammes | |
| Standard 26 gauge | 88 ± 3.0 grammes | |
| Standard 23 gauge | 130 ± 9.0 grammes | |

*Tip Force = force at the point of penetration of film by tip
^{@}Ramp force = peak force recorded at the change in needle diameter of DepotOne

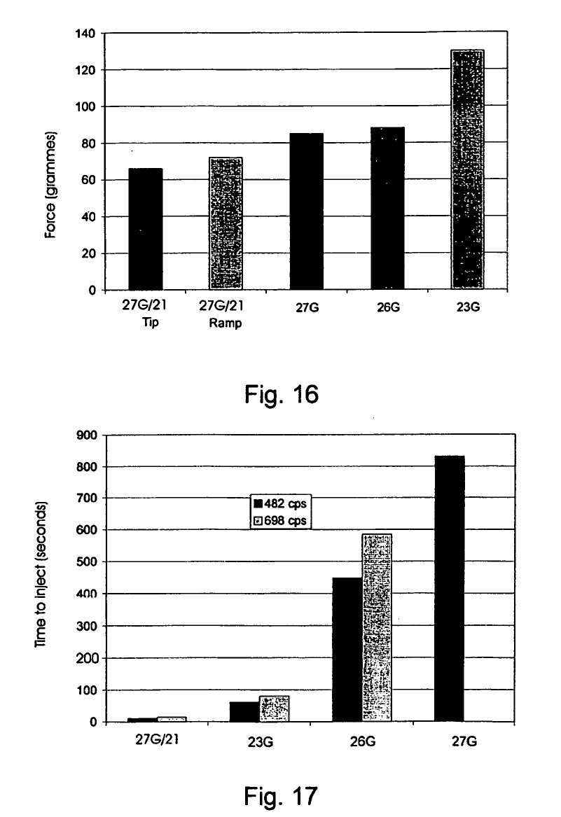

The graphical printout of FIG. 16 in the 27G/21 needle shows the detailed force fingerprint verses linear position of the needle.

The average tip penetration force for the 27G/21 needle into 0.4 mm Melab film was 67 grammes. The average ramp penetration force for 27G/21 needle into 0.4 mm Melab film was 72.1 grammes.

The 27G/21 needle had a penetration force characteristics similar to the smaller 27 and 26 gauge needles and substantially smaller than the larger 23 gauge.

### EXAMPLE 2

The flow of fluids through the 27G/21 needle and standard needles was compared.

Materials

Needles

27G/21 needle was as in Example 1 length This setup will give the required 12 mm skin penetration depth when used with a commercial autoinjector.
Standard 23G needle length 12.5 mm protruding from glass (6.5 mm depth with autoinjector)
Standard 26G needle length 15.5 mm protruding from glass (9.5 mm depth with autoinjector)
Standard 27G needle length 12.5 mm protruding from glass (6.5 mm depth with autoinjector)
   The comparative needles were standard needles.

All needles were glued into standard 1 ml glass syringes supplied by Form a Vitrum

Fluid Characterisation

Standard fluids were supplied by Brookfield Instruments.
The viscosity of the silicon oils was independently calibrated.
Temperature 22° C. (degree Celsius)
Relative humidity 58%
   Protocol
Fluid (1 ml) was loaded into glass syringe with needle attached
Syringe equilibrated in water bath
Syringe loaded in Flow analyser
10 N force was applied to syringe plunger.
Time for complete delivery of fluid in to air was assessed

The results are shown in Table 2.

TABLE 2

| | | Time of Delivery | | |
|---|---|---|---|---|
| | | (sec) ± standard deviation | | |
| | | Brookfield | Brookfield | |
| Needle | No. of tests | Silicone 482 cps | Silicone 698 cps | |
| 27G/21 needle | 10 | 10.5 ± 0.5 | 13.9 ± 0.4 | |
| 23 Gauge | 3 | 61. ± 1.9 | 80. | — |
| 26 gauge | 1 | 449. | 585. | — |
| 27 gauge | 1 | 832. | — | |

The results are shown graphically in FIG. 17 the new needle is the 27G/21 needle.

The 27G/21 needle delivered 482 centipoises (cps) standardised fluid in 10.5 seconds 10 and 698 cps fluid in 13.9 seconds. The 27G/21 needle was substantially faster than 23, 26 and 27 gauge standard needles.

### EXAMPLE 3

### Pain Test

A small uncontrolled volunteer study was carried out. This study indicated that the 27G/21 needle had a pain score, as measured by a Visual Analogue Scale (VAS), similar to a 26 gauge needle. Both these needles had a VAS score approximately half that of a 21 gauge needle. Similar results were found when the 27G/21 needle was used with a commercial autoinjector and with a manual syringe.

It was found that user perceptions of the 27G/21 needle, including visual perception and pain perception, became increasingly favourable (compared to a conventional needle of equivalent fluidic capacity) when the tip outer diameter was 25% to 50% smaller than the barrel outer diameter. This was achieved without reducing the fluidic capacity which was equivalent to or better than a 21G needle.

1-72. (canceled)

73. A needle for use with means to inject or remove material comprising (i) a tip which tapers to a point, (ii) an elongate section adjacent the tip, said elongate section comprises one or more ramps and optionally a parallel section, (iii) a barrel section in fluid connection with the means to inject/withdraw material, there being an inner bore comprising a fluidic pathway, which inner bore is within the barrel and optionally extends partially or fully down the elongate section and/or to the tip, there being one or more apertures fluidically connecting the said inner bore to the outside of the needle, and at least part of at least one aperture is lateral or on or adjacent to the barrel and/or the elongate.

74. A needle according to claim 73 in which the tip has a cutting end with one or more bevels sharpened into blades.

75. A needle according to any one of claims 73 in which the needle has a non cutting atraumatic tip, smooth and with a conical or curved tip with substantially curved radii at the foremost point so that, in use it substantially parts tissue without cutting.

76. A needle according to any one of claims 73 in which the tip is a sharp, partially sharp or non sharp blade of cross sectional area smaller than that of the elongate section.

77. A needle according to claim 73 in which the needle tip has an outside diameter of about 0.2 to 5.0 mm.

78. A needle according to claim 73 in which the needle length is 2 to 200 mm.

79. A needle according to claim 73 in which the needle tip diameter is 1% to 99% of barrel diameter

80. A needle according to claim 73 in which the ratio of the tip diameter to the barrel diameter is between 1:1.01 and 1:100

81. A needle according to claim 73 in which the ratio of the fluidic capacity of the barrel to the tube equivalent of the tip is between 1.05:1 to 1000:1.

82. A needle according to claim 73 in which at least part of the elongate section has a ramp where the effective outside dimension is made to increase gradually and/or in one or more steps from the tip until it joins the barrel section

83. A needle according to claim 78 in which the overall angle of one or more step/gradient and the angle and/or radius of the leading and trailing edges of step/gradient is chosen to ease entry with minimum force and/or pain/trauma of the skin/tissue being penetrated.

84. A needle according to claim 73 in which the elongate section has a fluid carrying bore or channel connecting fluidically to an aperture at tip and the bore or channel is of o, c, u, x, shape etc.

85. A needle according to claim 73 in which at least part of at least one aperture is spaced away from the tip and/or closer to the barrel than the tip.

86. A needle according to claim 73 in which the barrel has an outside diameter of 0.2 to 25 mm.

87. A needle according to claim 73 in which the bore and/or outside diameter of the barrel and elongate section and/or tip varies.

88. A needle according to claim 87 in which the bore and/or outside diameter changes at the connection between the barrel and the fluid holding or dispensing part.

89. A needle according to claim 87 in which all or part of the barrel section is inside the connection to a fluid container and made such that the majority of the greater than average bore of the needle does not penetrate the skin, but conducts the fluid to the point where the bore does penetrate the skin.

90. A needle according to claim 87 in which the elongate section is hollow or solid and has a wall thickness greater or lesser than the barrel

91. A needle according to claim 73 in which, in use, the elongate section maintains or increases the size of a path in tissue formed initially by the tip, and eases and directs the flow of fluid at least partly along or around the outside of the needle at least partly in a forward direction towards the tip.

92. A needle according to claim 73 in which, in use, when fluid pressure is applied, the elongate section forms all or part of a channel which nucleates or eases and directs the passage of at least part of the fluid flow with a smaller pressure drop than if the fluid were constrained inside the bore of the tube in that same location.

93. A needle according to claim 73 in which the aperture encompasses 10% or more of the circumference of the bore of the barrel.

94. A needle according to claim 73 in which the aperture is in the form of a groove or slot.

95. A needle according to claim 73 in which the one or more apertures extend in length more than 5 diameters of the adjacent tube and have an aspect ratio of more than 5:1 length to depth.

96. A needle according to claim 73 in which the one or more apertures have an included angle of less than 12° and the aperture(s) are positioned and angled to induce fluid to flow along and adjacent to the elongate section and are positioned and angled and radiused to reduce catching, cutting or coring of particulate fluid or skin or tissue in use.

97. A needle according to claim 73 in which at least part of at least one aperture is away from the tip and/or on a section of larger outer dimension than the tip.

98. A needle according to claim 73 in which the overall dimension of at least one aperture is larger than the tip outer dimension by a factor within the range of 10% or more.

99. A needle according to claim 73 in which the one or more aperture(s) are longitudinal or radial, round or elongate or a slot or a groove, and can penetrate through one wall or through both walls of the tube.

100. A needle according to claim 73 in which the aperture is in the form of a slot which is deformable and expands under pressure in use

101. A needle according to claim 73 in which at least part of at least one aperture is spaced apart from the tip and is located substantially where the there is a gradient or step change in the outside diameter.

102. A needle according to claim 101 in which one or more apertures is on or adjacent to a gradient or step change in outer dimension other than the tip.

103. A needle according to claim 73 in which at least part of gradient is positioned away from the heel of aperture at syringe end of aperture i.e. on opposite side of aperture from the tip.

104. A needle according to 73 in which at least part of at least one aperture is located on the gradient where the elongate section joins the barrel.

105. A needle according to claim 73 in which at least part of at least one aperture is located on the elongate section and optionally can or cannot not adjoin the tip.

106. A needle according to claim 73 in which the aperture is greater than 1-10 mm long

107. A needle according to claim 73 in which the aperture nearest the tip is spaced apart from the tip by more than about 0.5 mm.

108. A needle according to 73 in which the aperture has an aspect ratio of width to length in the range 1:1 to 1:500

109. A needle according to claim 73 in which the aperture has an aspect ratio of width to length greater than 1:10

110. A needle according to claim 73 in which the aspect ratio of the inner bore diameter to slot length is greater than or in range of 1:2 to 1:50

111. A needle according to claim 73 in which the aspect ratio of the inner bore diameter to slot length is greater than 1:5.

112. A needle according to claim 73 in which the aspect ratio of the outer tube diameter to slot length is greater than or in range 1:2 to 1:50

113. A needle according to claim 73 in which the aspect ratio of the outer tube diameter to slot length is greater than 1 to 4.

114. A needle according to claim 73 in which the ratio of slot length to overall needle length is greater than or in range 1:1.5 to 1:10

115. A needle according to claim 73 in which the end of the aperture nearest the tip is open.

116. A needle according to claim 114 in which the barrel enters the skin and the elongate section is solid and the aperture at the end of the barrel adjoins to the groove or slot and the groove or slot goes to the tip end of the needle

117. A needle according to claim 73 in which the end of the channel or bore nearest the tip is closed.

118. A needle according to claim 117 in which the needle is a solid needle and the aperture is a groove or slot and the groove or slot terminates at a position spaced apart from the tip.

119. A needle according to claim 73 which can incorporate a balloon catheter which comprises a deformable or elastic sheath which has an aperture or apertures in it, which sheath fits around or encases the needle and in use the aperture or apertures in the sheath are below the skin and connect with the aperture in the needle.

120. A needle according to claim 73 in which the needle has a length of 2 to 200 mm.

121. A needle according to claim 73 in which there is overall a minimum (from approximately 10% to 90%) of cutting and displacement by the needle itself in relation to the fluidic capacity of an equivalent conventional needle.

122. A needle according to claim 73 in which there is a minimum total dimension (length and or area from approximately 10% to 90%) of sharp cutting edges and/or points, and minimum level of sharpness in relation to the size and fluidic capacity of an equivalent conventional needle

123. A needle according to claim 73 in which in use the small aperture formed in skin and tissue by the tip is smoothly extended along the tip and elongate section with minimum of sharp edges or sharp changes in direction to catch or snag tissue

124. A needle according to claim 73 which comprises at least one separate component made of one or more different materials and which the separate parts are joined together

125. A needle according to claim 73 which is made by process steps comprising forming a step in the diameter of a needle tube, closing the tip of needle, forming a primary angle onto tip, processing by mechanical or chemical means to increase the radii of the edges of the tip and aperture, forming step to make final tip dimensions

126. A process of making a needle which needle is according to claim 73 which process includes the steps of providing surface texture to reduce friction and/or retain lubricant at surface before and during use.

127. A process of making a needle which needle is according to claim 73 which process includes increasing the edge radii of the tip and/or heel and/or edges of one or more apertures to prevent catching or snagging of fluid contents and/or skin.

128. An injection device incorporating a needle according to claim 73.

129. A needle and injection device according to claim 128 in which the injection device comprises a syringe, pen, autoinjector, syringe driver, tissue/fluid extraction device.

130. A needle according to claim 73 in which the length of the needle is chosen for the application of the needle.

131. A needle according to claim 130 in which the applications are selected from the use of the needle to add or remove substance including intradermal, sub-cutaneous, intra-muscular, intra-venous, into bone, into joint, into eye, into any organ or space, and for keyhole surgery.

132. A needle according to claim 73 in which there is a concave curvature on the face of tip and/or on other parts of needle and/or with ridges, rails or splines to reduce force/friction of needle against skin/tissue and/or coring of tissue into aperture.

133. A needle according to claim 132 in which the depth of the concave section is 1-80% of adjacent needle outer diameter.

134. A needle according to claim 132 in which the depth of the concave section is 1-80% of adjacent needle diameter and the concave section is radiused to 1-200% of outside diameter

135. A needle according to claim 73 in which there is a needle guide surrounding the needle which restricts the axial or lateral movement of needle and/or liquid holding elements attached to needle.

136. A needle according to claim 135 in which the guide restricts needle transaxial or lateral movement in use to below 0.01-1°; 1-10°; 0.1-1 mm; 1-3 mm.

137. A needle according to claim 135 in which the guide has one or more sprung elements to maintain tight tolerance while allowing for variations in manufactured size and guide has minimum contact with moving needle-holding element to reduce friction.

138. A needle according to claim 73 combined with an autoinjector or pen.

139. A needle according to claim 138 in which the autoinjector or pen has high pressure delivery of fluid in the range 0.1-200 bar or more.

140. A needle according to claim 73 in which the part of the needle barrel where the fluid first meets the constriction at the opening of the barrel has an entry-aperture of smaller inner dimension than the bore of the barrel for example included angle is below 90° or is of inverted funnel shape, whereby in use the first entry-aperture encountered by the fluid is of smaller inner dimension than the bore of the barrel.

141. A needle according to claim 73 in which the needle barrel projects into the injector or syringe so that it is proud of the end of the injector or syringe.

142. A needle according to claim 141 in which the needle barrel projects a distance of more than 1 mm from the bottom of the hub or injector or syringe barrel.

143. A needle according to claim 141 in which the distance from the needle barrel to the adjacent sidewalls of the hub or syringe or injector barrel is more than 0.1 mm.

144. The use of a needle according to claim 73 to add or remove substance or device for medicinal or diagnostic or other purposes for human or animal or other applications.
